(19) United States
(12) Patent Application Publication
(10) Pub. No.: US 2022/0265874 A1
(43) Pub. Date: Aug. 25, 2022

(54) AUTOMATIC SURFACE DISINFECTION

(71) Applicant: Innovasonic, Inc., Dublin, CA (US)

(72) Inventor: Boris Kobrin, Dublin, CA (US)

(21) Appl. No.: 17/389,054

(22) Filed: Jul. 29, 2021

Related U.S. Application Data
(60) Provisional application No. 63/151,772, filed on Feb. 21, 2021.
(60) Provisional application No. 63/188,389, filed on May 13, 2021.

(51) Int. Cl. A61L 2/08; A61L 2/24

(52) U.S. Cl. CPC A61L 2/085; A61L 2/24; A61L 2202/11; A61L 2202/14

(57) ABSTRACT

An apparatus, system and computer program product for disinfecting a touch surface, comprising a structure having a touch surface and a heating element thermally coupled to the touch surface. A controller is coupled to the heating element, wherein the controller is configured to control heating power supplied to the heating element in a manner that heats the touch surface to a sufficient temperature for a sufficient period of time sufficient to sanitize the touch surface from viruses and microorganisms.

### CLAIM OF PRIORITY BENEFIT

This application claims the priority benefit of commonly-assigned, co-pending U.S. provisional patent application No. 63/151,772, filed 21 Feb. 2021, entitled “Touch Surface Disinfection”, Attorney Docket No. WTC-2021-01-PR, and co-pending U.S. provisional patent application No. 63/188,389, filed 13 May 2021, entitled “Touch Surface Disinfection”, Attorney Docket No. ISI-012-PR, the entire disclosures of which are incorporated herein by reference.

### FIELD OF THE INVENTION

Aspects of the present are related to methods and devices for disinfecting surfaces, especially touch surfaces, specifically aspects of the present disclosure are related to the heat disinfection of touch surfaces.

### BACKGROUND OF THE INVENTION

The dominant method used today for cleaning and disinfection of touch surfaces is periodic manual cleaning by service personnel using chemicals and wipes. This process is done on a schedule, not frequent enough to cut spread of contamination. It does not guarantee spread of infection does not happen between cleanings, since even one contagious person touch event would contaminate surface and start spreading infection to other people. To eliminate a possibility of such spread touch screen must be disinfected after each customer.

There are also antibacterial coatings and antimicrobial glass products. Both are not irrelevant for the task of eliminating the spread of contamination since it takes 3-4 hours for these technologies to drop pathogens concentration to acceptable level (LOG3-4 reduction).

There is also a UV light disinfection technology, which has been used mostly in medical facilities with lamps; again, it is performed on schedule, once a day, at most.

Some companies have been trying to use UV technology on touch screens in the form of flood illuminators attached to a display frame. Since UV is harmful to people's skin and eye safety has been a limiting factor for commercialization of this technology.

It is well known that heat kills pathogens. For example, microbiologists who placed coronavirus samples on the interior surfaces of a car, including carpeting and plastic parts, found that viral concentrations were reduced by greater than 99% within 15 minutes when the interior air was heated to 133° F. (56° C.), raising surface temperatures to 120° F.

WHO guidelines on thermal disinfection of coronavirus is 15 min at 56° C. (133° F.)

Review Medical Virology journal published the following references:

Proven COVID-19 disinfection by heat has been reported in scientific articles (John Abraham et al, “Review Medical Virology,” April 2020):
3 minutes at 75° C. (160 F)
5 minutes at 65° C. (149 F)
20 minutes at 60° C. (140 F)

Recent tests with SARS-CoV-2 in Hong Kong showed that it became undetectable after five minutes at 158° F. (70° C.). The time required to kill SARS-CoV-2 increased as the temperature was reduced, such that the time by which it was undetectable increased to 30 minutes at 132° F. (56° C.), two days at 98.6° F. (37° C.), and two weeks at 71.6° F. (22° C.). At 39° F. (4° C.) the virus remained detectable at two weeks when the experiment ended (Chin et al, “Lancet” 2020).

Another example of bacteria incapacitation is disinfection of food. UHT pasteurization of milk can be done at high temperatures (140° C.) over very short times, e.g., 1-2 s (H. Deeth et al., “Improving the Safety and Quality of Milk: Milk Production and Processing,” 2010) Thus, there is a great need to provide disinfection to surfaces people touch during everyday life. The most obvious surfaces susceptible to transmitting infection are shared cars infotainment systems, ATM displays, interactive kiosks, vending machines, self-check stations in airports, elevator control screens, doorknobs, handrails, etc.

### BRIEF DESCRIPTION OF THE DRAWINGS

The teachings of the present disclosure can be readily understood by considering the following detailed description in conjunction with the accompanying drawings, in which:

FIG. 1 depicts a three dimensional side view of an apparatus for disinfecting a touch surface according to aspects of the present disclosure.

FIG. 2 depicts a three dimensional cross section view of another implementation of the apparatus for disinfecting a touch surface according to aspects of the present disclosure.

FIG. 3A shows a side view of an implementation of a self-disinfecting touch surface according to aspects of the present disclosure.

FIG. 3B depicts a three dimensional side view of an implementation of the self-disinfecting touch surface of FIG. 3A coupled to a mounting surface according to aspects of the present disclosure.

FIG. 4A depicts a top down view of a capacitive touch screen configured as an apparatus for disinfecting a touch surface according to aspects of the present disclosure.

FIG. 4B shows a three dimensional side view of a capacitive touch screen configured as an apparatus for disinfecting a touch surface according to aspects of the present disclosure.

FIG. 5A depicts a three dimensional view of a resistive touch screen configured as an apparatus for disinfecting a touch surface according to aspects of the present disclosure.

FIG. 5B depicts a three dimensional view of a self-disinfecting touch surface that can be retrofit to another device according to aspects of the present disclosure.

FIG. 6 depicts the apparatus for disinfecting a touch surface with localized heating after a touch event.

FIG. 7 depicts yet another embodiment of the apparatus for disinfecting a touch surface with induction power according to aspects of the present disclosure.

FIG. 8 is a three dimensional view that depicts another alternative implementation of the apparatus for disinfecting a touch surface with electromagnetic radiation power according to aspects of the present disclosure.

FIG. 9 depicts a three dimensional cut away view of another alternative implementation of the apparatus for disinfecting a touch surface with electromagnetic radiation power according to aspects of the present disclosure.

FIG. 10 is a three dimensional cut away view of another alternative implementation of the apparatus for disinfecting a touch surface with electromagnetic radiation power according to aspects of the present disclosure.

FIG. 11A depicts a side view of an escalator handrail belt having an alternative implementation of the apparatus for disinfecting a touch surface according to aspects of the present disclosure.

FIG. 11B shows a side view of another implementation of the escalator handrail belt having the apparatus for disinfecting a touch surface according to aspects of the present disclosure.

FIG. 12A depicts a side view of a doorknob with cut-away portions of an implementation of the apparatus for disinfecting a touch surface according to aspects of the present disclosure.

FIG. 12B shows a side view of another implementation of a doorknob having the apparatus for disinfecting a touch surface according to aspects of the present disclosure.

FIG. 13A depicts a cut away side view of a handrail having an implementation of the apparatus for disinfecting a touch surface according to aspects of the present disclosure.

FIG. 13B depicts a cut away side view of another implementation of a handrail incorporating apparatus for disinfecting a touch surface according to aspects of the present disclosure.

FIG. 14 depicts a heating controller system for implementing touch surface sanitization according to aspects of the present disclosure.

FIG. 15 is a flow diagram depicting a method for touch surface sanitization from viruses and other microorganisms according to aspects of the present disclosure.

FIG. 16 is a flow diagram depicting another method for touch surface sanitization from viruses and other microorganisms according to aspects of the present disclosure.

FIG. 17 is a flow diagram depicting another method for touch surface sanitization from viruses and other microorganisms according to aspects of the present disclosure.

### DESCRIPTION OF THE SPECIFIC EMBODIMENTS

Although the following detailed description contains many specific details for the purposes of illustration, anyone of ordinary skill in the art will appreciate that many variations and alterations to the following details are within the scope of the invention. Accordingly, the exemplary embodiments of the invention described below are set forth without any loss of generality to, and without imposing limitations upon, the claimed invention.

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the figure(s) being described. Because components of embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

In the interest of clarity, not all of the routine features of the implementations described herein are shown and described. It will be understood by those skilled in the art that in the development of any such implementations, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of the present disclosure.

In accordance with aspects of the present disclosure, the components, process steps, and/or data structures may be implemented using various types of operating systems; computing platforms; user interfaces/displays, including personal or laptop computers, video game consoles, PDAs and other handheld devices, such as cellular telephones, tablet computers, portable gaming devices; and/or general purpose machines. In addition, those of ordinary skill in the art will recognize that devices of a less general purpose nature, such as hardwired devices, field programmable gate arrays (FPGs), application specific integrated circuits (ASICs), or the like, may also be used without departing from the scope and spirit of the inventive concepts disclosed herein.

The rise of touch screen devices has lead to an increased contact with surfaces, touch screen terminals such as those found in ATMs, gasoline station pumps and vending machines may come in to contact with hundreds of individuals per day. The surface of the touch screen may become a breeding ground for bacteria and other organisms. An effective way to eliminate bacteria and other organisms is via heat. The ability to eliminate viruses, bacteria, and other organisms using heat may be produced on new devices, retrofitted onto existing touch screen devices or applied to existing devices through a software or firmware update according to aspects of the present disclosure.

An apparatus, system and computer program product for disinfecting a touch surface, comprising a structure having a touch surface and a heating element thermally coupled to the touch surface. A controller is coupled to the heating element, wherein the controller is configured to control heating power supplied to the heating element in a manner that heats the touch surface to a sufficient temperature for a sufficient period of time sufficient to sanitize the touch surface from viruses and microorganisms. The controller may be further configured to control the heating power supplied to the heating element to sanitize and concurrently maintain the touch surface at a temperature that safe to be touched. In some implementations, the controller may be further configured to control the heating power supplied to the heating element to sanitize the touch surface and then return the touch surface to a temperature that is safe to be touched.

The structure may comprise a substantially transparent insulation layer disposed between a substantially transparent heating element and the touch surface, wherein the touch surface is an exposed surface of the substantially transparent insulation layer. An adhesive layer may be disposed underneath the structure wherein the adhesive layer is substantially transparent when attached to a mounting surface. The heating element may be a resistive wire heating element. The resistive wire heating element may further include a substantially transparent array of resistive heating wires coupled to a substrate and arranged on a surface of the substrate. The structure having the touch surface may include the substrate. In some implementations, the heating element may be located on a front side of the touch surface and the heating element may further comprise a substantially transparent insulator coupled to a side opposite the touch surface. In other implementations, the heating element may be located on a side of the structure having a touch surface facing the inside of a device housing.

The structure having a touch surface may include a touch screen. The touch screen may be a capacitive touch screen and the heating element includes a capacitive touch sensing electrode in the touch screen. Alternatively, the touch screen may be a resistive touch screen and the heating element may include an electrode of the resistive touch screen. In some implementations, the structure having a touch surface is a handrail or handrail belt of an escalator. In some other implementations, the structure having a touch surface is doorknob, door handle or car-door handle.

The heating element may be configured to receive the heating power through electromagnetic induction. The heating element may be configured to receive the heating power by radio frequency induction, the heating element may further comprise an array of substantially transparent antennas arranged on a surface of a substrate and the controller may include a radio frequency transmitter. Different areas of the substrate may have transparent antennas configured to resonantly receive different radio frequencies. Each of the different areas of the substrate may be thermally coupled to a corresponding different area of the touch surface and the controller may be further configured to transmit a radio frequency corresponding to a radio frequency of antennas in an area of the touch surface where a touch has event has concluded. Alternatively, the heating element may include transparent electrically conductive material and the controller may include an induction coil configured to induce a current in the transparent electrically conductive material.

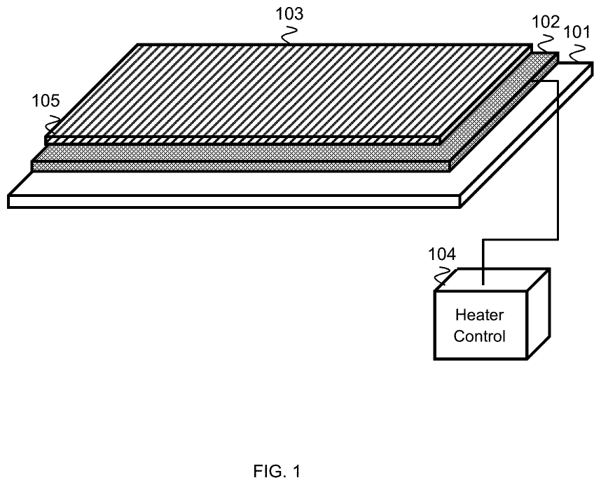

FIG. 1 depicts a three dimensional side view of the apparatus for disinfecting a touch surface according to aspects of the present disclosure. As shown, the apparatus comprises a heating element 102 thermally coupled to a structure 105 having a touch surface 103. Located underneath the heating element 102 is a mounting surface 101. The heating element 102 may be embedded, laminated, formed in, or otherwise affixed to the mounting surface 101. The structure 105 may be an insulating layer formed on a surface of the heating element. The insulating layer may be a dielectric material such as glass, rubber, plastic, or similar non-conductive polymer. The heating element 102 may be any material or combination of material that produce heat when current flows in them. For example and without limitation the heating element 102 may be a resistive wire heating element. The resistive wire may be made of any known heating material such as, carbon, nickel, iron, chromium, tungsten, tantalum, copper, aluminum, indium, or any suitable alloy thereof. The resistive wire heating element may be an array of wires or other wire structure characterized by a wire width and wire spacing sufficient to be substantially transparent when arranged under the structure 105. By way of example, the resistive wire heating element may include an array of heating wires arranged at regular or semi-regular intervals. The combination of wire diameter and the interval between adjacent wires may be configured to produce a substantially transparent heating element. In some implementations, this substantially transparent heating may be in the form of a wire mesh. The structure having the touch surface may also be made of a substantially transparent material such a glass, plastic, aluminum. In the case of aluminum or other conductive material, the heating element may be embedded or encased in a transparent insulating substrate such as plastic or lacquer to prevent leakage of current. A heater controller 104 is coupled to the heating element. The heater controller 104 is configured to control the power supplied to the heating element 102 in a manner that heats the touch surface to a sufficient temperature and for a sufficient time period to sanitize the surface of the touch surface from viruses and microorganisms. The heater controller 104 may include circuitry and integrated circuit devices to control the power supply to the heating element 102. The heater controller 104 may also include a temperature sensor located near the touch surface. The temperature sensor may be for example and without limitation, a thermistor, thermocouple, bimetallic switch, or the like. The temperature sensor may be part of an integrated circuit device of the heater controller or another device near the structure having the touch surface. Alternatively, the temperature of the touch surface may be determined from the change in resistance in the heating wire thermally coupled to the touch surface. This determination may use the known temperature dependent resistance of the heating wire to determine the temperature of the touch pad surface. The equation T=(1/α) (R/R₀−1)+T₀ determines the current temperature (T) from the current resistance (R) and the standard temperature (T₀) resistance (R₀) and the known temperature coefficient of resistance of the material (α).

The sufficient temperature and sufficient time to sanitize the touch surface from viruses and microorganisms may be determined experimentally. As discussed above clinical experimentation has shown that the COVID-19 virus is destroyed on a surface held at 70 C for five minutes. Other studies have shown that 90% of active E. coli is destroyed in 1.65 Minutes at 65 C, (see V. Juneja “Thermal destruction of Escherichia coli O157:H7 in beef and chicken: determination of D- and Z-values,” Int. J. of Food Microbiology, Vol. 35 Iss. 3 p. 231-237 (1997)). Other virus and microorganism destruction temperatures and times may be determined experimentally via growth of viral or microorganism populations in growth media and exposure of test populations to varying temperatures for varying amounts of time. Linear regression and curve fitting, population modeling may further be used to refine population destruction temperatures and times.

According to aspects of the present disclosure the touch surface may be heated according to different modes. For example, in some implementations the touch surface may be continuously heated continuous to a temperature acceptable for human touch, e.g., with no interruption in touch screen operation. In alternative implementations, referred to herein as pulse heating, the touch surface may be rapidly heated to a high temperature that is sufficient for disinfection but higher than acceptable for human touch and then rapidly cooled to safe-to-touch temperatures. In such implementations, the apparatus may include a cooling mechanism to facilitate rapid cooling. Such a cooling mechanism may be operatively coupled to the controller 104. Examples of such cooling mechanisms include convective cooling mechanisms (e.g., a fan), evaporative cooling mechanisms, e.g., a mechanism that sprays a mist of water or alcohol, conductive cooling, Peltier effect cooling, radiative cooling, or some combination of two or more of these that operate in response to signals from the controller.

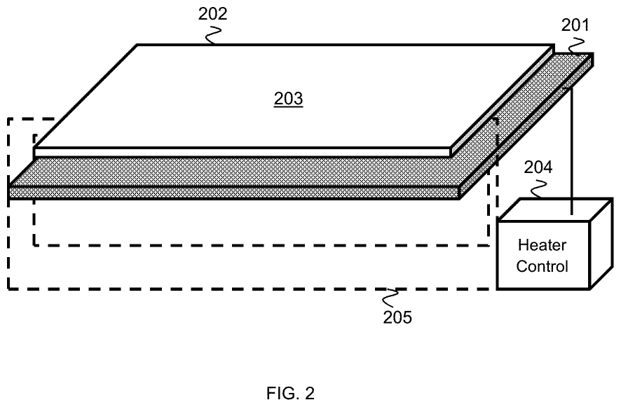

FIG. 2 depicts a three dimensional cross section view of another implementation of an apparatus for disinfecting a touch surface according to aspects of the present disclosure. In this implementation, the heating element 201 is thermally coupled to a rigid structure 203 having the touch surface 202. Here the structure 203 provides support to the heating element 201, which may be glued, epoxied, clipped, or otherwise affixed to a bottom of the structure 203 on the side opposite the touch surface 202. Alternatively, the heating element may comprise metal traces formed on the bottom of the structure 203. The heating element may a conductive metal such as copper tin, indium, aluminum, gold, silver or an alloy such as indium tin oxide (ITO) or the like. The touch surface 202 may be for example and without limitation, user facing side of a touch screen, or the user facing side of an interface such as a button. As such, the structure 203 may include elements of a touch screen such as a display, touch digitizer, cabling, glass panel, plastic panel or clear metallic panel. The heating element 201 may be coupled to to the non-user facing side of the structure 203 and heat from the heating element is thermally coupled to the touch surface 202 through the structure 203. The heating element may be located on the side of the structure 203 facing an inside of a housing 205. The housing 205 may be for example and without limitation a cellphone housing, a tablet computer housing, a touch monitor housing or any other type of device structure configured to protect device components. A controller 204 is coupled to the heating element 201 and is configured to control the power supplied to the heating element in a manner that heats the touch surface to sufficient temperature for a sufficient period of time to sanitize the touch surface from viruses and microorganisms. The controller 204 may be connected to a power supply and include transistors, switches, relays arranged to control the supply of power to the heating element 201. In some implementation the controller may include other power supply control components such as a, AC/DC converter, boost converter or buck converter.

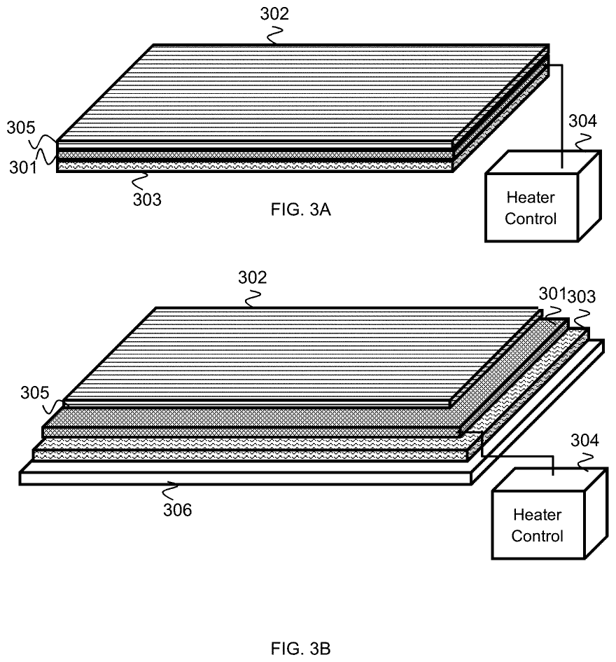

FIG. 3A shows a side view of an implementation of the apparatus for disinfecting a touch surface according to aspects of the present disclosure. Here, the apparatus is shown without a mounting surface. In this implementation, the structure 305 having the touch surface 302 may be comprised of a flexible insulating material such as, glass, plastic, rubber, or other polymer. Additionally, the structure 305 may be substantially transparent to facilitate use with a touch screen or user interface buttons. The heating element 301 may also be substantially transparent. The heating element may be a wire heater having an array of wires or metal traces. Alternatively, the heating elements may include arrays of structures made of other electrically conductive materials, such as ITO, silver nanowires (AgNW), graphene, Poly(3,4-ethylenedioxythiophene) (PEDOT), or carbon nanotubes (CNT). The structures in the array are characterized by a width and a spacing between adjacent wires configured to render the array substantially transparent. By way of example, and not by way of limitation, the width may be between 0.5 microns and 10 microns and the spacing may be between 30 microns and 500 microns. Here, a substantially transparent means greater than 80% visible light transmission. Additionally the heating element 301 may include multiple wires or metal traces arranged in a pattern underneath the structure 305. The space between wires or metal traces is sufficient to create a substantially transparent heating element. An adhesive layer 303 may be located underneath the heating element 301. In some implementations, the adhesive layer 303 may also be coupled to the structure 305 through spaces in the heating element 301. In this implementation, the adhesive layer 303 may affix the heating element 301 to the structure 305. To ensure a substantially transparent apparatus the refractive index of the cured adhesive layer may be chosen to match the refractive index of the structure 305. A heater controller 304 is coupled to the heating element 301. As discussed above, the heater controller is configured to control heating power supplied to the heating element in a manner that heats the touch surface to a sufficient temperature for a sufficient period of time sufficient to sanitize the touch surface from viruses and microorganisms. The heater controller may include a power supply 304 such as a battery to power the heating element independently from any device or surface the apparatus is mounted.

Turning to FIG. 3B the apparatus of 3A is shown attached to a mounting surface 306. The adhesive 303 may be configured to be substantially transparent when in contact with the mounting surface 306. Further, the adhesive 303 may be chosen such that its refractive index matches the refractive index of the material of the mounting surface. For example and without limitation if the mounting surface 306 is Eagle XG® display glass with a refractive index n of about 1.51 then a substantially transparent adhesive having a similar refractive index such as cyanoacrylate (n≈1.49), Poly-vinyl Chloride (PVC) tape (n≈1.54) or optical adhesive may be used. Eagle XG® is a registered trademark of Corning Incorporated of Corning, N.Y. The mounting surface 306 may be a touch screen and the controller may be configured to interrupt, disable or otherwise decouple the supply of heating power to the heating element after the touch surface has been sanitized to avoid interference with operation of the touch screen. In some implementations, the adhesive may include spacers that prevent direct contact of the heating element with the mounting surface 306. In such an implementation, the adhesive layer may not cover the entire bottom surface of the heating element allowing a gap between the heating element and the mounting surface for better touch detection.

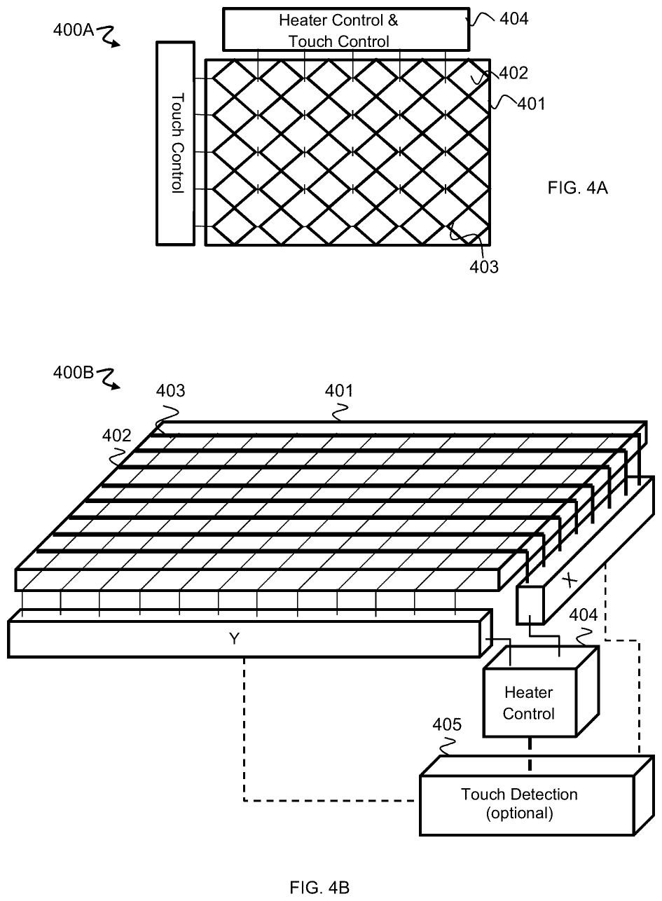

FIG. 4A depicts a top down view of a capacitive touch screen 400A configured as a self-disinfecting touch surface according to aspects of the present disclosure. As shown, the touch screen includes a structure 401 having a touch surface (not shown). Embedded in the structure 401 or underneath the structure 401 is an array of capacitance sensing electrodes. The array of capacitive electrodes are arranged in X rows 402 and Y columns 403. Here, the heating element is integrated into the capacitance sensing electrodes, e.g., by configuring the Y columns 403 of sensing electrodes to act as the heating elements. The touch sensing electronics coupled to the capacitive sensing electrodes may also be configured to act as the heater controller 404, e.g., by suitable, hardware, firmware, or software modification. The heater controller 404 may control the power to the Y columns 403 to generate heat that travels through the structure to the touch surface. The heater controller 404 is configured to control heating power supplied to the heating element in a manner that heats the touch surface to a sufficient temperature for a sufficient period of time to sanitize the touch surface from viruses and microorganisms. In some implementations, heater controller 404 may be configured to apply heating power to selected Y columns to localize the heating power supplied to the structure 401 and thereby preferentially heat a limited portion of the touch surface.

FIG. 4B shows a three dimensional side view of a thin film similar to that shown in FIG. 3A configured as a self-disinfecting touch surface 400B according to aspects of the present disclosure. As in FIG. 4A, a thin film structure 401 includes conductors arranged in X columns 402 and Y columns 403. A heater controller 404 may be configured to apply heating power to selected X and/or Y columns to localize the heating power supplied to the structure 401 and thereby preferentially heat a limited portion of the touch surface. In some implementations, the heater controller 404 may be configured to interoperate with an optional touch controller 405 that is separate from the heater controller 404. The touch controller 405 may be communicatively coupled to the heater controller 404. The communicative coupling of the touch controller 405 and the heater controller 404 allows for region limited heating of the touch surface 401. In the region limited heating operation schema, regions of the touch surface that have recently been touched and after the touch event ends, may have power applied to them to heat up the touch electrodes local to the touch event. Localizing the heating to the area a touch event occurred allows for reduced energy consumption and a reduced burn danger. The heater controller 404 may include power control for each of the electrodes to allow for individual control and for heating power to be applied at controlled locations of the touch surface. The controller may be configured to control heating power supplied to the heating element in the area where the touch event occurred in a manner that heats the area of the touch surface where the touch event occurred to a sufficient temperature for a sufficient period of time to sanitize the touch surface from viruses and microorganisms. A touch event may be an intersection a finger or other body part with the touch surface.

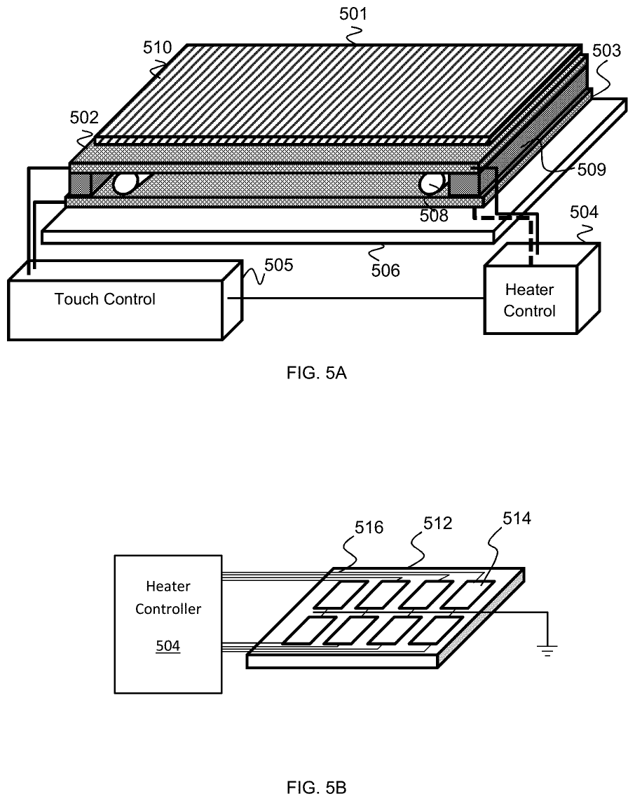

FIG. 5A depicts a three dimensional view of a resistive touch screen configured as an apparatus for disinfecting a touch surface according to aspects of the present disclosure. As shown the resistive touch screen apparatus includes a structure 510 having a touch surface 501. Underneath the structure 510, may be a top electrode 502 and a bottom electrode 503 separated by a spacer 508 and an insulator 509. This structure may be disposed on a mounting surface 506 for example without limitation a glass, plastic or metal surface. The heater controller 504 may control power to the top electrode 502 or the bottom electrode 503 or both. During operation, the controller may increase the power supplied to the one or more of the electrodes to cause resistive heating. Additionally the heater controller 504 may be communicatively coupled to the touch screen controller 505.

FIG. 5B depicts a top plan view of a self-disinfecting touch surface 511 that may be retrofit to an existing device, such as a touch screen or touch interface to provide localized disinfection by way of resistive heating. The self-sanitizing touch surface 511 includes a generally planar structure 510, e.g., a sheet of flexible transparent film into which an array of conductive patches 514 and conductive traces 516 has been integrated. The conductive traces 516 are configured to provide selective coupling of electrical power from a heating controller 504 to each conductive patch. As discussed above with respect to capacitive touch screens, communication with the touch screen controller may allow the heater controller 504 to localize heating of the touch surface 501 to areas of the touch screen where a touch event has finished occurring. Here, the conductive patches 514 act as resistive electrodes that serve as the heating elements for the self-disinfecting touch surface. The heater controller 504 is configured to control heating power supplied to the heating element in a manner that heats some or all of the area of the touch surface 501 to a sufficient temperature for a sufficient period to sanitize the touch surface from viruses and microorganisms.

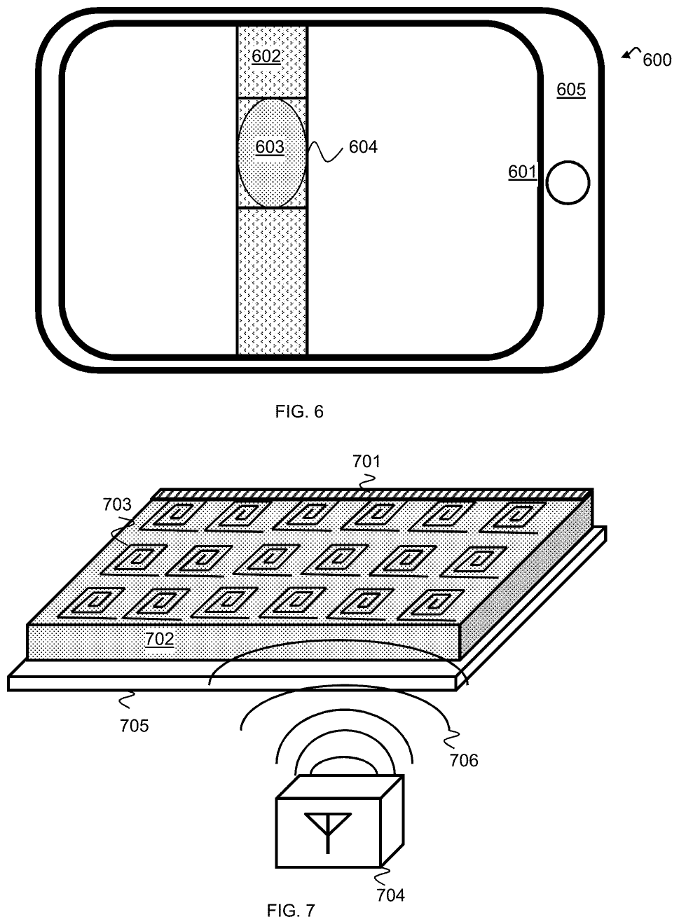

FIG. 6 depicts an apparatus for disinfecting a touch surface with localized heating after a touch event. As shown, the apparatus is integrated into a touch sensitive device 600 such as a smartphone or touch capable monitor. The device 600 has a touch screen that includes the apparatus. The front of the touch screen is also the touch surface 601. A touch event 603 may occur in an area 604 of the touch screen 601. This area 604, where the touch event 603 occurred is detected by the touch screen controller using either capacitive electrodes or resistive electrodes, depending on the touch screen type. The heater controller may receive touch event information from the touch controller and use the touch information to localize heating of the touch surface 601. The heating in this embodiment is localized 602 to a strip of the touch surface 601 this is due to the layout of capacitive touch sensing electrodes that are also used as the heating element. As seen more clearly in FIG. 4A, the electrodes run vertically and horizontally through the device and heating may be localized in either direction in long strips that run across the touch surface 601. Additionally in the implementation shown, heating elements are located on a side of the structure having a touch surface 601 facing the inside of a housing 605. Here, the housing creates a bezel around the touch surface 601 and runs underneath the structure having a touch surface with a cavity for the device components including the apparatus.

FIG. 7 depicts yet another non-limiting example of an apparatus for disinfecting a touch surface with induction power according to aspects of the present disclosure. Here, the structure having a touch surface 701 is disposed upon a substrate 702. The substrate 703 includes one or more antennas 703 formed on or in the substrate. The mounting surface 705 may be disposed underneath the substrate 702. Alternatively, the one or more antennas 703 may be coupled to the back of the structure having a touch surface 701. It should be noted that, to prevent electrocution the structure having a touch surface may be comprised of an insulating material such as plastic, rubber or glass. The heater controller 704 may include an electromagnetic (e.g. Radio frequency (RF)) transmitter that transmits power 706 to the antenna heating elements 703. The one or more of the Antennas 703 transduce the electromagnetic signal inducing a current flow through the antenna. The antenna may also include a resistive heating wire or alternatively the antenna 703 may itself be configured to heat the touch surface. In some implementations, the one or more antennas may include at least two antennas that transduce different radio frequencies. These at least two different antennas may be located at different of the touch surface 701. The heater controller 704 may be configured to generate the different electromagnetic frequencies of the antennas to heat different areas of the screen.

By way of example, and not by way of limitation, each antenna 703 may be in the form of a flat coil that can resonantly receive electromagnetic radiation, e.g., radiofrequency radiation. Each antenna may be configured to have a different resonant frequency, e.g., by varying the inner and out diameters of the coils, the number of windings in the coils, the width of each winding or some combination of two or more of these. Such configuration allows a given heating element 703 to be selectively and preferentially heated by subjecting the array of heating elements to electromagnetic radiation having a frequency corresponding to the resonant frequency of the given heating element.

Alternatively, the heater controller 704 may be configured to generate an alternating electromagnetic field, for example and without limitation the heater controller 704 may include or be coupled to a motor that drives a spinning magnet or an alternating current (AC) source that drives an induction coil. In this alternative implementation, the one or more antennas 703 may be simple sheets of conductive material. The alternating magnetic field generates eddy currents in the conductive material causing it to heat up. The conductive material may be substantially transparent.

The heating element, of which the one or more antennas are a part, is thermally coupled to the touch surface. Thus, the antennas heat the touch surface from power controlled by the heat controller 704. The properties of the antenna may be used to determine the power of the electromagnetic signal or the speed of the alternating magnetic field required to heat the touch surface to a sufficient temperature for a sufficient period to sanitize the touch surface from viruses and microorganisms.

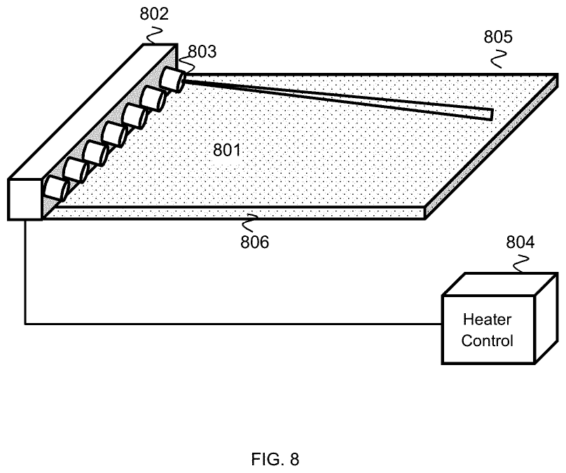

FIG. 8 is a three dimensional view that depicts another alternative implementation of an apparatus for disinfecting a touch surface with electromagnetic radiation power according to aspects of the present disclosure. In this implementation the electromagnetic radiation thermally couples a heating element to the touch surface. In this implementation the heating element 802 includes an array of electromagnetic radiation emitters 803 for example and without limitation infrared emitting diodes. The electromagnetic radiation emitters 803 are configured to project electromagnetic radiation (e.g. infrared light) on to the touch surface 801. The structure 801 having the touch surface 801 may be comprised of a material that absorbs the wavelength of electromagnetic radiation emitted by the heating element 802. For example and without limitation the material may be filtering glass, polycarbonate, etc. The structure 806 absorbs the electromagnetic radiation, transforming the energy of the radiation to heat, subsequently heating the touch surface. The touch surface 801 also absorbs some radiation and generates heat but generally, the thermal mass of the structure 806 underlying the touch surface is greater and contributes a majority of the heat to the touch surface 801.

The heater controller 804 may control the power supplied to the heating element 802 and may control the each of the electromagnetic radiation emitters 803, individually. The heat controller 804 may touch event information to localize sanitation to the area the touch event occurred. To localize sanitation the heater controller 804 may supply power only to the electromagnetic radiation emitters 803 that shine 805 on the area where the touch event occurred. The heater controller 804 is configured to control heating power supplied to the heating element 802 in a manner that heats the touch surface to a sufficient temperature for a sufficient period of time sufficient to sanitize the touch surface from viruses and microorganisms. The absorptive properties of the structure 806 and the emission capabilities of the electromagnetic radiation emitters 803 to determine the exposure length and power required to by the radiation emitter to heat the touch surface sufficiently.

Although a linear array of emitters 803 is shown in FIG. 8, aspects of the present disclosure are not limited to just such implementations. Specifically, aspects of the present disclosure include implementations in which a two-dimensional array of radiation emitters, e.g., light emitting diodes (LEDs) are incorporated into the structure 806 and configured to project the electromagnetic radiation through portions of the structure towards the touch surface 801. In some of these implementations, the heater controller 804 may be configured to selectively active a subset of one or more emitters in the array to localize the supply of radiation to the touch surface and thereby localize heating thereof.

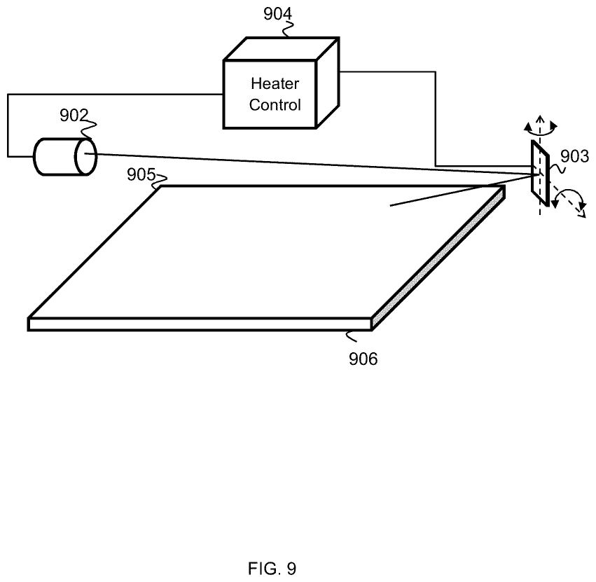

FIG. 9 depicts a three dimensional cut away view of another alternative implementation of the apparatus for disinfecting a touch surface with electromagnetic radiation power according to aspects of the present disclosure. In this implementation the electromagnetic radiation thermally couples the heating element to the touch surface 901. The heating element here uses a beam of electromagnetic radiation 905 to directly heat the touch surface 901. The electromagnetic radiation 905 may be for example and without limitation infrared, visible, or ultraviolet radiation. The heating element may comprise a suitable electromagnetic radiation emitter 902 and a scanner 903. The electromagnetic radiation 905 from the emitter 902 may be focused into a beam using optics and scanned across the touch surface by the scanner 903. In some implementations, the radiation emitter 902 may be an LED or a laser, e.g., a a laser diode, fiber laser, solid-state laser, semiconductor laser or the like that supplies coherent radiation. The scanner 903 is configured to scan the electromagnetic radiation 905 across the touch surface 901. Scanning may involve moving emitter itself or, alternatively, by directing the radiation 905 onto a mirror, which may rotate about one or more axes, e.g., vertical and horizontal axes of rotation). The scanner may be for example and without limitation a macro scale device, such as a galvanometer scanning mirror, a motorized mirror, or a motorized prism. Alternatively, the scanner 903 may be a micro electromechanical system (MEMS) device. The scanner 903 directs the electromagnetic radiation 905 to hit the touch surface 901. The area of the touch surface 901 hit by the electromagnetic radiation 905 may absorb energy from the radiation and quickly heat up thereby sanitizing the area.

The electromagnetic radiation may be scanned across the entire touch surface to sanitize the entire surface. Alternatively, touch event data may be used by the heater controller 904 to control power to the scanner 903 to direct the coherent electromagnetic radiation 905 to an area where the touch event has occurred. The heater controller 904 may control power to both the electromagnetic radiation emitter 902 and the scanner 903 to steer the coherent electromagnetic radiation and control the intensity of the beam. The heater controller 904 is further configured to control radiative power supplied by the radiation emitter 902 and/or scanning by the scanner in a manner that heats the touch surface 901 to a sufficient temperature for a sufficient period to sanitize the touch surface from viruses and microorganisms.

Although a single emitter and scanner are shown in FIG. 9, implementations that utilize two or more scanners and emitters are within the scope of aspects of the present disclosure. In some such implementations emitters and scanners may be located along two or more different edges of the touch surface.

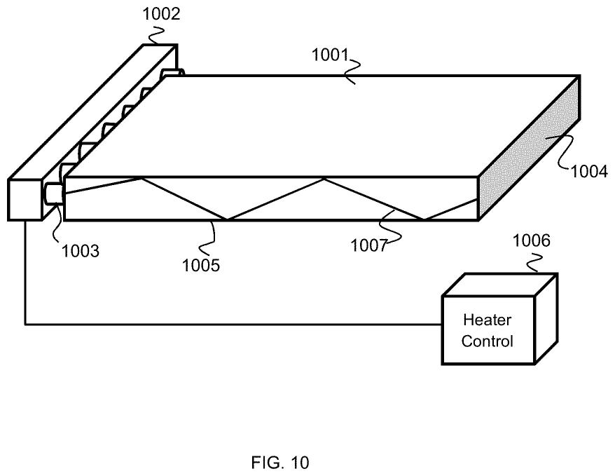

FIG. 10 is a three dimensional cut away view of another alternative implementation of the apparatus for disinfecting a touch surface with electromagnetic radiation power according to aspects of the present disclosure. In this implementation, the heating element 1002 comprises one or more electromagnetic radiation emitters 1003 arranged on a side of the structure 1004 having a touch surface 1001 and focused into a minor surface of the structure perpendicular to the major surfaces. The touch surface 1001 may include a material that is reflective to the frequency of electromagnetic radiation of the heating element. On a major side of the structure, 1004 opposite the touch surface 1001 another material reflective of the frequency of electromagnetic radiation of the heating element 1002. The radiation 1007 is directed into the structure 1004 and reflects off the reflective major surfaces 1001, 1005 of the structure 1004, heating the structure and touch surface 1001. The controller 1006 is configured to control heating power supplied to the heating element 1002 in a manner that heats the touch surface 1001 to a sufficient temperature for a sufficient period of time to sanitize the touch surface from viruses and microorganisms. The controller 1006 may control the intensity and length of ‘on’ state time of the electromagnetic radiation emitters 1003 to control the temperature of the touch surface 1001. The electromagnetic radiation emitters may be for example and without limitation infrared lights. The structure 1004 may be comprised of a material that is transparent for visible light wavelengths and absorptive of the wavelength of the electromagnetic radiation 1007. For example and without limitation the structure may be made from polycarbonate, filter coated glass or the like.

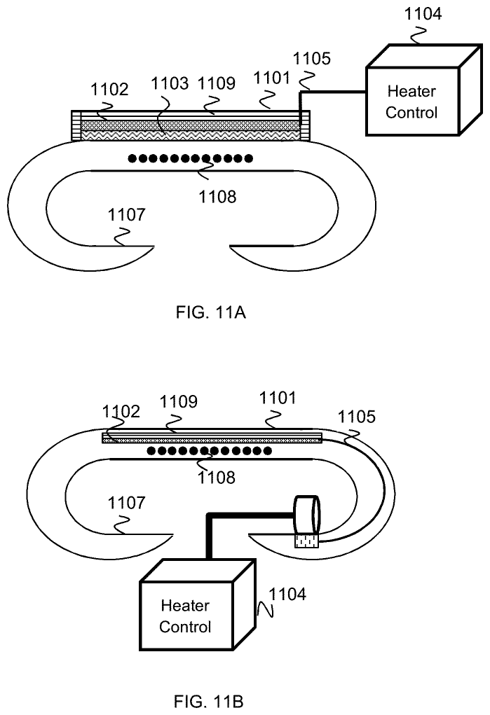

FIG. 11A depicts a side view of drawing of an escalator handrail belt having an alternative implementation of the apparatus for disinfecting a touch surface according to aspects of the present disclosure. Typically, escalators include a handrail that moves with the step via a belt system sliding across a stationary track. Escalator riders grab on to the handrail to stabilize themselves while using the escalator. The handrail belt movement is timed with the movement of the stairs so that the rider can remain stationary and hold the rail up and down the escalator. As such, the escalator handrail belt 1107 is frequently in contact with user's hands and therefore subject to potential microbial contamination. The escalator handrail belt includes metal bands 1108, which run the length of the belt and provide rigidity to the belt. The Apparatus in this implementation may be affixed to the handrail belt via an adhesive layer 1103. The handrail 1107 is the mounting surface. The structure 1109 having a touch surface 1101 may be an insulating layer disposed on top of a heating element 1102. An adhesive layer 1103 may be disposed underneath the heating element 1102. The adhesive layer 1103 may attach the heating element 1102 and the structure 1109 to the escalator handrail belt 1107. Note that this implementation is similar to the stand-alone implementation shown in FIG. 3A. The structure 1109 in this implementation may slightly differ from the one shown in FIG. 3A in that it includes sidewalls that surround and electrically insulate the heating element 1103.

A heater controller 1104 is coupled to the heating element 1102, e.g., by a coupling element 1105 and is configured to control heating power supplied to the heating element 1102 in a manner that heats the touch surface 1101 to a sufficient temperature for a sufficient period of time to sanitize the touch surface from viruses and microorganisms. The heating controller 1104 may be coupled by any of the disclosed methods shown in FIGS. 1-7.

The heater controller in some implementations may be small enough to fit underneath the heating element or near a portion of the structure. FIG. 11B shows another implementation of the escalator handrail belt having the apparatus for disinfecting a touch surface according to aspects of the present disclosure. In this implementation, the apparatus is integrated into the escalator handrail belt 1107. Here the structure 1109 having a touch surface 1101 is integrated into the body of the belt 1107 above the heating element 1102. The heating element may be coupled to the heater controller 1104 by a coupling mechanism 1105 in the form of a wire that runs to the underside of the belt 1107. A roller and contact pad may ensure that the heater controller 1104 is in electrical contact with the wire and can supply heating power to the heating element 1102 in the form of electric current.

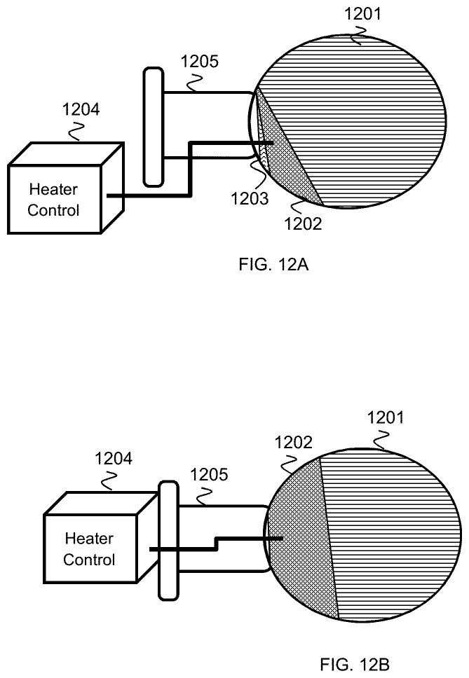

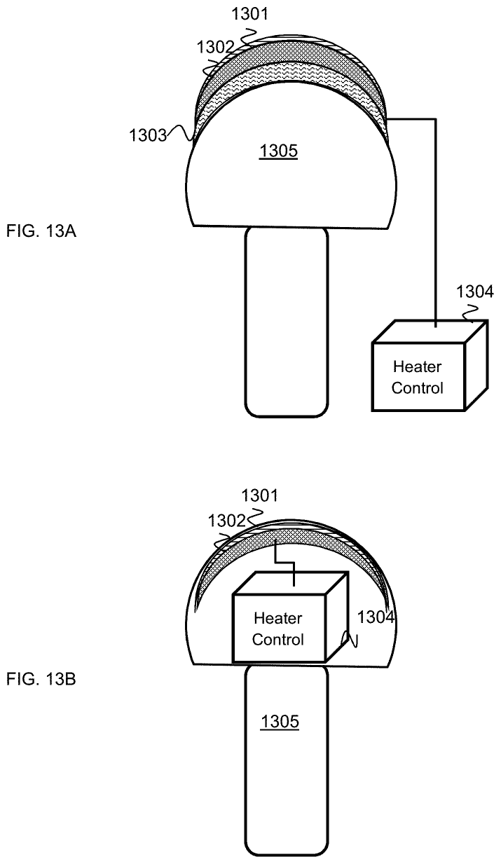

FIG. 12A depicts a side view of a doorknob with cut-away portions of an implementation of the apparatus for disinfecting a touch surface according to aspects of the present disclosure. Here an apparatus similar to the device shown in FIG. 3A, is attached to the doorknob 1205 with an adhesive layer 1203. The heating element 1202 is disposed over the adhesive layer 1203 and the structure having the touch surface 1201 is located on top. The heating element 1202 is thermally coupled to the touch surface 1201 by conduction through close physical proximity to the touch surface. A heating controller 1204 is coupled to the heating element 1202 and is configured to control heating power supplied to the heating element 1202 in a manner that heats the touch surface 1201 to a sufficient temperature for a sufficient period of time to sanitize the touch surface from viruses and microorganisms. The heating controller may be integrated into the heating element or small enough to fit underneath the heating element in the adhesive layer. The heater controller 1204 may be coupled by any of the above-disclosed methods in FIGS. 1-7. FIG. 12B shows another implementation of a doorknob having the apparatus for disinfecting a touch surface according to aspects of the present disclosure. Here the heating element 1202 and structure having the touch surface 1201 is integrated into the doorknob 1205. The heating controller 1204 may also be coupled to the heating element 1202 inside the doorknob 1205 and the heating controller itself may, in some implementations, be integrated into the doorknob.

FIG. 13A depicts a cut away side view of a handrail having an implementation of the apparatus for disinfecting a touch surface according to aspects of the present disclosure. Here an apparatus similar to the device shown in FIG. 3A, is attached to the handrail 1305 with an adhesive layer 1303. The heating element 1302 is disposed over the adhesive layer 1303 and the structure having the touch surface 1301 is located on top. The heating element 1302 is thermally coupled to the touch surface 1301 by conduction through close physical proximity to the touch surface. A heating controller 1304 is coupled to the heating element 1302 and is configured to control heating power supplied to the heating element 1302 in a manner that heats the touch surface 1301 to a sufficient temperature for a sufficient period of time to sanitize the touch surface from viruses and microorganisms. The heating controller may be integrated into the heating element or small enough to fit underneath the heating element in the adhesive layer. The heater controller 1304 may be coupled by any of the above disclosed methods in FIGS. 1-7. FIG. 13B depicts another implementation of a handrail having the apparatus for disinfecting a touch surface according to aspects of the present disclosure. Here the heating element 1302 and structure having the touch surface 1301 is integrated into the handrail 1305. The heating controller 1304 may also be coupled to the heating element 1302 inside the handrail 1305 and the heating controller itself may, in some implementations, be integrated into the handrail.

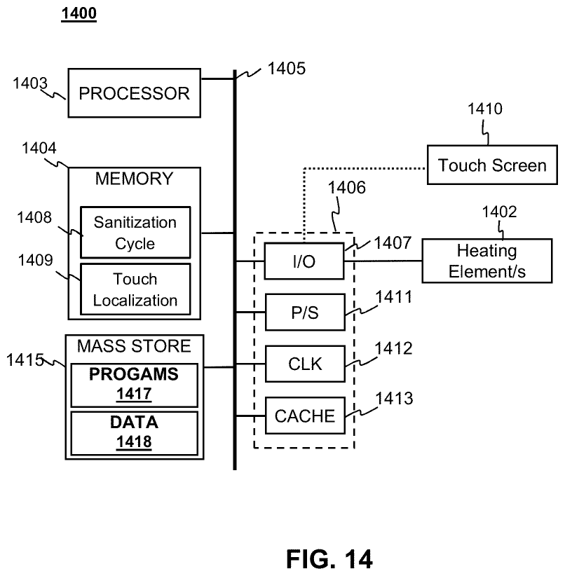

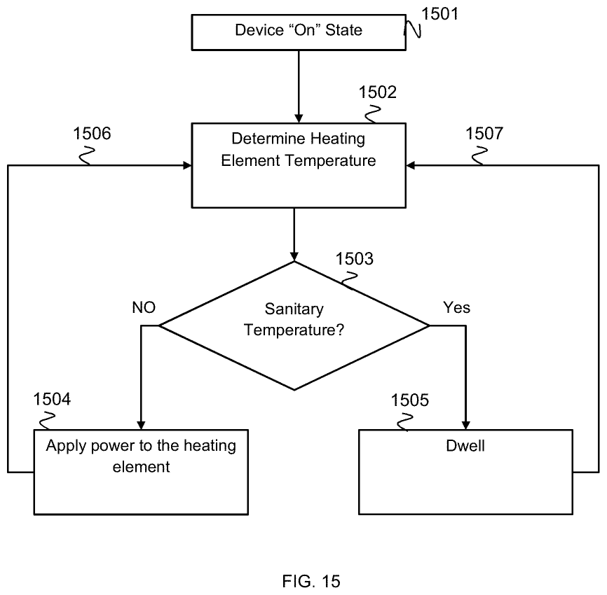

FIG. 14 depicts a heating controller system for implementing touch surface sanitization methods like that shown in Figures throughout the application, for example those described below with respect to FIGS. 15-17. The system may include a computing device 1400 coupled to a heating element 1402 and (optionally) a touch screen 1410. The heating element 1402 may also be coupled to the touch screen 1410. The touch screen 1410 may alternatively be a touch screen controller. The heating element may include temperature-sensing devices as discussed above.

The heater controller 1400 may include one or more processor units 1403, which may be configured according to well-known architectures, such as, e.g., single-core, dual-core, quad-core, multi-core, processor-coprocessor, and the like. The computing device may also include one or more memory units 1404 (e.g., random access memory (RAM), dynamic random access memory (DRAM), read-only memory (ROM), and the like).

The processor unit 1403 may execute one or more programs, portions of which may be stored in the memory 1404 and the processor 1403 may be operatively coupled to the memory, e.g., by accessing the memory via a data bus 1405. The programs may include touch surface sanitization cycle methods 1408 such as those shown in FIGS. 15-17. Additionally, the Memory 1404 may store touch screen decoding and touch screen localization information 1409 this information may be used to decode and utilize information from the touch screen 1410 or a touch screen controller. The Touch screen localization 1409, Touch surface sanitization cycle methods 1408 may be stored as data 1418 or as programs 1417 in the Mass Store 1418.

The processor unit 1403 is further configured to execute one or more programs 1417 stored in the mass store 1415 or in memory 1404, which cause the processor to carry out the one or more of the methods described above.

The computing device 1400 may also include well-known support circuits, such as input/output (I/O) 1407, circuits, power supplies (P/S) 1411, a clock (CLK) 1412, and cache 1413, which may communicate with other components of the system, e.g., via the bus 1405. The computing device may include a network interface 1414. The computing device may optionally include a mass storage device 1415 such as a disk drive, CD-ROM drive, tape drive, flash memory, or the like, and the mass storage device may store programs and/or data.

FIG. 15 is a flow diagram depicting a method for touch surface sanitization from viruses and other microorganisms according to aspects of the present disclosure. This implementation of the sanitization method maintains the touch surface at a temperature sufficient to kill viruses and microorganisms on the touch surface while still being at a temperature safe for brief touches. Initially the apparatus may be in an ‘off’ state. From the ‘off’ state a signal or power to the heating controller may put the device in an ‘on’ state at 1501. In the ‘on’ state the heater controller determines the heating element temperature and by thermal coupling, the touch surface temperature at 1502. The determination of heating element temperature may be performed using heating element resistance or a thermometer, thermocouple or other temperature-sensing element near the heating element or touch surface, as discussed above. The heater controller checks if the heating element or touch surface is at a sanitary temperature at 1503. This may be performed by comparing the measured temperature to a known sanitary temperature stored in memory. For example and without limitation the sanitary temperature may be 70 C as discussed above.

If the touch surface or heating element is not at a sanitary temperature then the power is applied through the heating element at 1504. This may be accomplished by switching relay, transistor or other type of switch between a power supply and the heating element. Alternatively, power may be applied to the heating element in any of the other alternative methods discussed above. After power is applied to the heating element for a heating interval (for example and without limitation 5 s, 10 s, 15 s etc.) the method returns at 1506 to determining the heating element or touch surface temperature at 1502 and the method continues checking if the heating element or touch surface temperature at 1503 is at a sanitary temperature.

If the heater controller determines that the heating element or touch surface temperature the controller goes into a dwell state at 1505 wherein the heating element is allowed to cool and additional power is not applied to the heating element for the dwell period. The dwell period may be an experimentally determined amount of time that the touch surface or heating element may maintain temperature without receiving additional power. After the dwell period ends the method returns at 1507 to determining the touch surface or heating element temperature at 1502 and the method loops again.

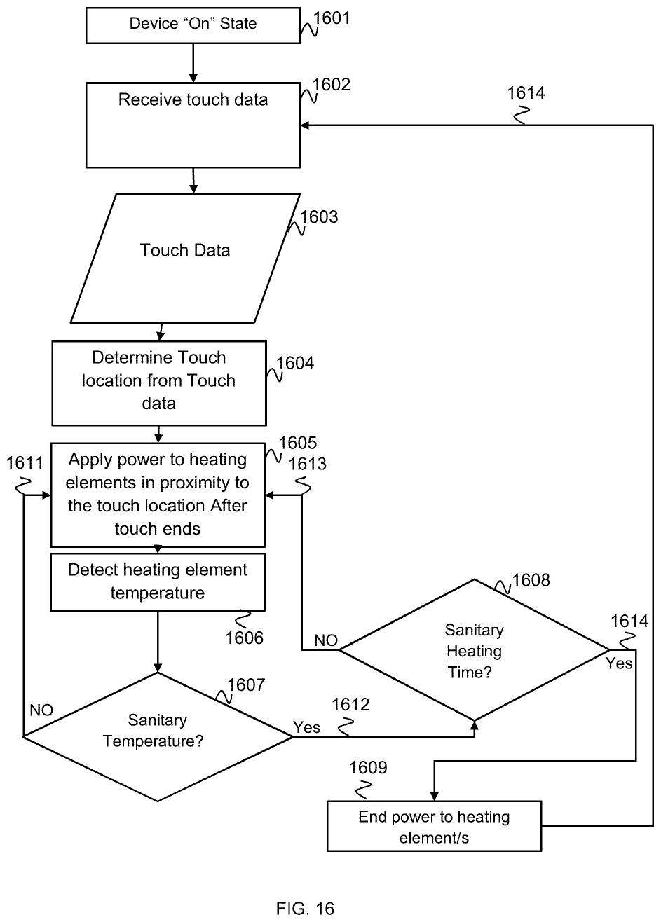

FIG. 16 is a flow diagram depicting another method for touch surface sanitization from viruses and other microorganisms according to aspects of the present disclosure. In this implementation, the heating element or touch surface is heated after a touch event ends. The controller initially may be put into an ‘on’ state at 1601 from and ‘off’ state or a power saving mode. At 1602, the controller may receive touch data 1603 from a touch controller or in the case of an integrated heat controller and touch controller the heat controller may detect the touch event and determine the touch location. The touch location on the touch screen may be determined from the touch data at 1604. The controller may apply power to the heating elements in proximity to the touch location on the touch surface at 1605 after the touch event ends. A table in memory may associate heating element locations to touch screen locations determined from touch data. Note that in this implementation heat is applied after the touch event is detected to have ended to ensure that the user is not hurt by the hot touch surface. The current or power applied to the heating element may be applied for heating interval and then the heating element or touch surface temperature is detected at 1606. The temperature may be detected using any method discussed above. The controller then determines whether the touch surface or heating element is at a sanitary temperature at 1607. When the touch screen or heating element is not at a sanitary temperature, the method returns at 1611 to applying power to the heating element at 1605.

When it is determined that the heating element or touch surface is at a sanitary temperature at 1612 the controller checks if the heating element or touch surface has maintained the sanitary temperature for a sanitary heating time at 1608. The controller may trigger at timer when the touch surface or heating element initially reaches the sanitary temperature and reset the timer after the sanitary time has been reached. As discussed above the sanitary time may be for example and without limitation 5 minutes at 70 C. When the sanitary time has not been achieved, at 1613 the controller continues to apply power to heating element 1605 and the method starts over. When the controller determines that heating time has been met at 1614, heating power is removed from the heating element 1609 and the method begins again at 1615.

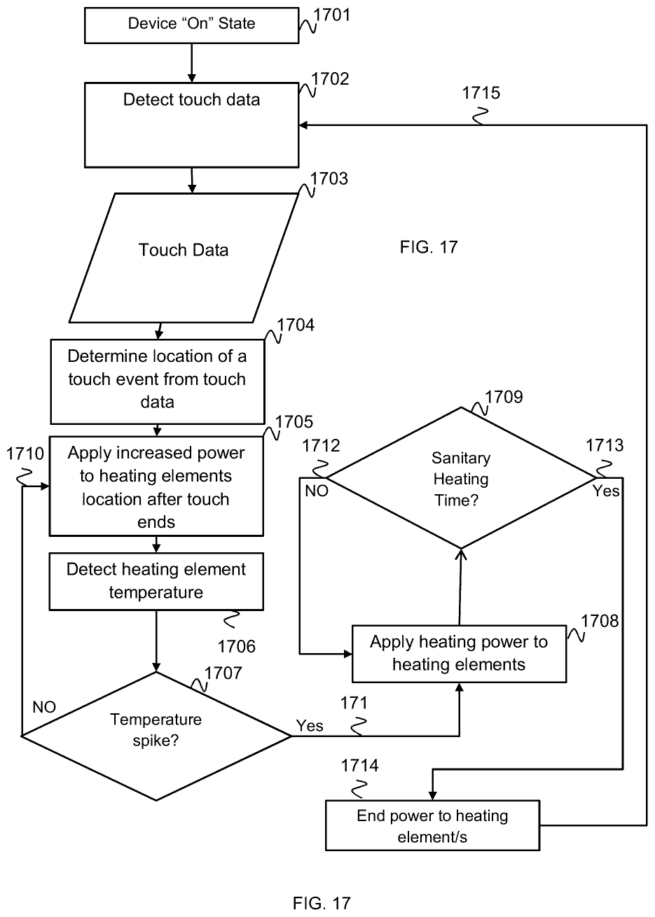

FIG. 17 is a flow diagram depicting another method for touch surface sanitization from viruses and other microorganisms according to aspects of the present disclosure. After being put into an ‘on’ state at 1701, the controller waits for touch data. Touch data may be detected at 1702 such as for example and without limitation when touch data is received from a touch screen controller or the touch heater controller detects touch data from a touch screen. The heater controller may use the touch data 1703 to determine a location a touch event occurred at 1704. In some implementations if localization of heating is not possible and the entire heating area will be heated after a touch event ends. In other implementations, only the areas of the touch surface in proximity to where the touch event occurred will be heated. Here, a large amount of power is applied to the heating elements at 1705 after a touch event ends to cause a large spike in temperature and quickly disinfect the surface. The temperature of the touch surface is detected after a heating interval at 1706. The heating interval may be chosen experimentally as a time it takes for the heating element to heat the touch surface to a high sanitary temperature at from room temperature. The controller monitors the temperature and determines if the touch surface or heating element temperature has spiked to the high sanitary temperature at 1707. The high sanitary temperature may be a threshold stored in memory. When the touch surface or heating element has not achieved the high sanitary temperature threshold, the controller continues at 1710 applying increased power to the heating elements at 1705.

When the touch surface or heating element temperature has spiked past the high sanitary temperature threshold at 1711 the power to the heating elements is reduce to a lower heating level at 1708. The heater controller monitors the temperature of the heating element or touch surface and applies the power to the heating elements to ensure that heating element or touch surface maintains a sanitary temperature. After the initial temperature spike, a timer may be started on the heating controller. The heater controller checks the timer every lower level heating interval to determine whether the sanitary heating time has expired at 1709. The lower level heating interval may be experimentally determined as an amount time for an amount power to be applied to the heating element to maintain the sanitary temperature. Additionally the sanitary time clock maybe restarted if it is determined that the temperature of the touch surface has dropped below the lower level sanitary temperature. If the sanitary timer has not expired, at 1712 power will continue to be applied to the heating element at 1708. If the sanitary timer has expired, at 1713 then power will cease to be applied to heating element at 1714 and the sanitation cycle is complete. The method will then restart at 1715 waiting for touch data at 1702.

While the above is a complete description of the preferred embodiments of the present invention, it is possible to use various alternatives, modifications, and equivalents. Therefore, the scope of the present invention should be determined not with reference to the above description but should, instead, be determined with reference to the appended claims, along with their full scope of equivalents. Any feature, whether preferred or not, may be combined with any other feature, whether preferred or not. In the claims that follow, the indefinite article “A” or “An” refers to a quantity of one or more of the item following the article, except where expressly stated otherwise. The appended claims are not to be interpreted as including means-plus-function limitations, unless such a limitation is explicitly recited in a given claim using the phrase “means for”. Any element in a claim that does not explicitly state “means for” performing a specified function, is not to be interpreted as a “means” or “step” clause as specified in 35 USC § 112, ¶6.

What is claimed is:

1. An apparatus for disinfecting a touch surface, comprising:
a structure having a touch surface;
one or more heating elements thermally coupled to the touch surface;
a controller coupled to the one or more heating elements, wherein the controller is configured to control heating power supplied to the one or more heating elements in a manner that heats the touch surface to a sufficient temperature for a sufficient period of time to sanitize the touch surface from viruses and microorganisms.

2. The apparatus from claim 1 wherein the structure comprises a substantially transparent electrically insulating layer disposed between one or more substantially transparent heating elements and the touch surface, wherein the touch surface is an exposed surface of the substantially transparent electrically insulating layer.

3. The apparatus from claim 1 wherein the one or more heating elements include an electrically resistive heating element.

4. The apparatus of claim 3, wherein the electrically resistive heating element includes one or more structures made of electrically conductive wire or metal traces or one or more structures made of indium tin oxide (ITO), silver nanowires (AgNW), graphene, Poly(3,4-ethylenedioxythiophene) (PEDOT), or carbon nanotubes (CNT).

5. The apparatus from claim 3 wherein the electrically resistive heating element includes a substantially transparent array of structures made of electrically conductive material coupled to a substrate and arranged on a surface of the substrate.

6. The apparatus of claim 1, wherein the one or more heating elements and the controller are configured to selectively supply heating power to a limited portion of the touch surface.

7. The apparatus of claim 1 wherein the one or more heating elements include one or more heating elements located on a front side of the touch surface and the heating element further comprises a substantially transparent insulator coupled to a side opposite the touch surface.

8. The apparatus of claim 1 wherein the one or more heating elements include one or more heating elements located on a side of the structure having a touch surface facing the inside of a device housing.

9. The apparatus of claim 1, wherein the one or more heating elements include one or more radiation emitters configured to project electromagnetic radiation toward the touch surface.

10. The apparatus of claim 1 wherein the one or more heating elements include one or more heating elements configured to receive the heating power through electromagnetic induction.

11. The apparatus of claim 10 wherein the one or more heating elements include one or more heating elements configured to receive the heating power by radio frequency induction and the heating element further comprises an array of substantially transparent antennas arranged on a surface of a substrate and the controller includes a radio frequency transmitter.

12. The apparatus of claim 11 wherein different areas of the substrate have transparent antennas configured to resonantly receive different radio frequencies, wherein each of the different areas of the substrate is thermally coupled to a corresponding different area of the touch surface and wherein the controller is further configured to transmit a radio frequency corresponding to a radio frequency of antennas in an area of the touch surface where a touch has event has concluded.

13. The apparatus of claim 10 wherein the one or more heating elements include one or more heating element that include transparent electrically conductive material and the controller includes an induction coil configured to induce a current in the transparent electrically conductive material.

14. The apparatus of claim 1 wherein the structure having a touch surface includes a touch screen.

15. The apparatus of claim 14 wherein the touch screen is a capacitive touch screen and the heating element includes a capacitive touch sensing electrode in the touch screen.

16. The apparatus of claim 14 wherein the touch screen is a resistive touch screen and wherein the heating element includes an electrode of the resistive touch screen.

17. The apparatus of claim 14 wherein the controller is configured to receive touch event information and localize heating of the heating element to areas of the touch screen where the touch event occurred after the touch event ends.

18. The apparatus of 1 wherein the structure having a touch surface is doorknob, door handle. a car-door handle, a handrail or handrail belt of an escalator.

19. The apparatus of claim 1 wherein the controller is further configured to control the heating power supplied to the one or more heating elements to sanitize and continuously maintain the touch surface at a temperature that is safe to be touched.

20. The apparatus of claim 1 wherein the controller is further configured to control the heating power supplied to the one or more heating elements in a pulsed mode to heat the touch surface to a temperature that is higher than a temperature that is safe to be touched and then return the touch surface to a temperature that is safe to be touched.
